(19) United States
(12) Patent Application Publication
(10) Pub. No.: US 2025/0089997 A1
(43) Pub. Date: Mar. 20, 2025

(54) BUTTON VALVE FOR ENDOSCOPIC TUBE SET

(71) Applicant: Boston Scientific Scimed, Inc., Maple Grove, MN (US)

(72) Inventors: Scott Edward Corbeil, Litchfield, NH (US); Ryan Vincent William Pollock, Sterling, MA (US)

(73) Assignee: Boston Scientific Scimed, Inc., Maple Grove, MN (US)

(21) Appl. No.: 18/889,582

(22) Filed: Sep. 19, 2024

Related U.S. Application Data
(60) Provisional application No. 63/584,010, filed on Sep. 20, 2023.

(51) Int. Cl. A61B 1/00; A61B 1/12

(52) U.S. Cl. CPC A61B 1/00128; A61B 1/00137; A61B 1/00142; A61B 1/125

(57) ABSTRACT

Methods and systems providing fluid flow to an endoscope. A container and tube set configured to couple to an endoscope may comprise a first container configured to contain a fluid, a first water supply tube including a first lumen extending therethrough and in fluid communication with the first container, a branched connector positioned in line with the first water supply tube and including a first fluid inlet, a first fluid outlet, and a second fluid outlet, a second container having a second fluid inlet in selective fluid communication with the second fluid outlet of the branched connector, a flow control valve positioned to obstruct flow between the second fluid outlet of the branched connector and the second fluid inlet of the second container, and a second water supply tube including a second lumen extending therethrough and in selective fluid communication with the bottom portion of the second container.

### CROSS-REFERENCE TO RELATED APPLICATIONS

This application claims the benefit of U.S. Provisional Patent Application Ser. No. 63/584,010 filed on Sep. 20, 2023, the disclosure of which is incorporated herein by reference.

### FIELD

This disclosure relates generally to medical fluid containers and methods, and particularly to a container and tube sets to supply fluid and/or gas to an endoscope.

### BACKGROUND

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. For example, sterile water may be used to irrigate the working lumen during the procedure. Further, during endoscopic procedures, the video lens at the distal end of the endoscope, which is used to navigate and visualize target tissues, may be prone to becoming fouled with blood, mucous, and other debris during the procedure. In order to reduce complications that may arise from removing the endoscope, manually cleaning the lens, and re-inserting (such as trauma or infection) nearly all endoscopes may be equipped with a lumen through which a cleaning fluid (which may typically be sterile water) can be delivered to the surface of the lens for the purpose of de-fouling.

The current state of the art for devices used to deliver sterile water to the endoscope for the purpose of irrigation and endoscope lens washing draw and/or push water from either one or two disposable bottles of sterile water (typically 1 L volume). In the two-bottle system, two separate disposable sterile water bottles may be used to supply sterile water separately to the endoscope for irrigation or lens washing. For irrigation, a flexible conduit may extend through a cap, which can be fitted onto the sterile water bottle after opening, and to the bottom of the bottle on the inlet end, may be inserted within the drive head of a peristaltic roller pump and connected to the endoscope through a scope specific connector. In the two-bottle system, a second sterile water bottle may be fitted with a cap having an inlet tube/connector which allows pressurized air or CO₂ gas to enter the bottle, and having a flexible conduit which extends from the bottle to, and connecting with, the lens wash port of the endoscope via a scope specific connector. Using this system, CO₂ gas may be supplied to the bottle at a pre-determined pressure, which in turn creates a pressure differential, driving sterile water through the conduit to the lens wash inlet of the endoscope, such that when the lens wash switch on the endoscope is triggered, sterile water jets through a dedicated lumen within the endoscope and washes sterile water over the fouled lens, thus clearing the lens.

In the one-bottle system, a cap may be attached to a single water bottle having a first conduit extending to the bottom of the bottle and extends through the roller pump and connecting to the irrigation port of the endoscope. A second conduit may extend to the bottom of the bottle and extend to the lens wash port on the endoscope. A third conduit may bring pressurized gas (e.g., air or CO2) into the bottle through a port on the cap. This system may allow a single sterile water bottle to be used for both lens wash and irrigation instead of a separate bottle for lens wash and irrigation.

Currently commercialized systems for delivering sterile water to the endoscope may rely on commercially available bottles of sterile water, with which the sterile water is pumped or pushed with compressed gas from the bottle to the scope. The volume of water available to the user is limited by the size/volume of commercially available sterile water bottles and space on procedure carts. In many hospital centers, the sterile water delivery systems may have a one-way valve at the end of the conduit to the scope to prevent backflow of fluids from the patient end of the scope back to the inlet sterile water conduit so that the sterile water delivery system may be used for multiple cases, over the course of a day.

An innovative design that can address many of the challenges and or limitations associated with current systems is a system which utilizes solution bags as the water source instead of using prefilled solution bottles. These solution bags are available in volumes including but not limited to 250, 500, 1000, 2000, 3000 and 4000 mL. This gives the user many different options for the initial setup volume of the water source, allowing the user to tailor the volume to their use needs. One embodiment of such a system is presented below. This system uses a spike port and septum valve connectivity design to connect a pre-filled solution bag with the endoscope tube set. Water from the primary solution bag flows to a “Y” in which part of the flow feeds the irrigation circuit and part of the flow feeds the lens wash circuit. Flow on the irrigation side of the “Y” flows through a flexible tubing conduit which is loaded into the head of an irrigation pump (typically a peristaltic pump) and continues to the endoscope umbilicus where it is connected via a threaded luer connector. Water on the lens wash side of the “Y junction flows into a secondary pressure vessel which can be subsequently pressurized on demand by the endoscope operator to create a pressure differential, which subsequently forces fluid from the lens wash pressure vessel to the umbilicus and ultimately to the lens wash nozzle at the end of the endoscope.

One of the challenges with this Design is that when internal pressure is applied to the lens wash pressure vessel, pressurized gas (air or CO2) will also pressurize the irrigation fluidic circuit, including the primary solution bag which is used to provide water to the tubing set for both irrigation and lens wash functions. Although the system as a whole will still function with this design, there are some potential drawbacks to this system, including but not limited to, pressurization of an off the shelf bag in which burst strength characteristics may vary greatly, bubbling of gas into the primary solution vessel may be concerning or distracting to the user, Pressurizing the system means that both irrigation and lens wash conduits will be pressurized and could lead to fluid spraying out when tubing is disconnected from the tubing sets, pressurization of the irrigation tubing set may cause the septum tube internal diameter to grow, reducing the retaining forces between the spike port and the septum tube (potentially for dislodgement and gross leakage).

One potential embodiment to prevent undesirable pressurization of the irrigation conduit is to add a one way valve to the design between the “Y” junction and the lens wash pressure vessel. Upon pressurization of the lens wash pressure vessel.

Pressurization of the pressure vessel would cause the one way valve to close, eliminating the potential for fluid to flow upstream to the primary solution bag, and thus eliminating the potential for pressurization of the primary solution bag and the irrigation circuit.

Another potential issue which is not solved by a one way valve placed between the “Y” connector and the lens wash pressure vessel is that when the tubing set is connected to the primary solution bag (via spiking of the solution bag), water will flow freely from the solution bag to the lens wash bag with nothing to stop the flow when the pressure vessel is full. Thus, left with this embodiment, water may continue to flow from the primary solution bag into and over flowing the lens wash pressure vessel and allowing water to flow up the air conduit of the umbilicus and into the pump circuit of the endoscope processor capital equipment. Which would definitely be undesirable.

Given the clear advantages of the aforementioned tubing set configuration over conventional systems, a need exists for improvements to the valving separating the two vessels.

### SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

In a first example, a container and tube set arranged and configured to couple to an endoscope for use in an endoscopic procedure, the container and tube set includes a first container configured to contain a fluid, the first container having a port in fluid communication with a bottom portion thereof; a first water supply tube including a first end, a second end, and a first lumen extending therethrough, wherein the first lumen is in fluid communication with the first container and the second end of the first water supply tube is positioned external to the first container; a branched connector positioned in line with the first water supply tube between the first and second ends thereof, the branched connector including a first fluid inlet, a first fluid outlet, and a second fluid outlet, a portion of the first lumen of the first water supply tube extending between the first fluid inlet and the first fluid outlet; a second container configured to contain a fluid, the second container having a second fluid inlet in selective fluid communication with the second fluid outlet of the branched connector; a flow control valve positioned to obstruct flow between the second fluid outlet of the branched connector and the second fluid inlet of the second container, the flow control valve movable between a closed position preventing fluid flow from the branched connector to the second container and preventing fluid flow from the second container to the branched connector and an open position permitting fluid flow from the branched connector to the second container; and a second water supply tube including a first end, a second end, and a second lumen extending therethrough, wherein the second lumen is in selective fluid communication with the bottom portion of the second container and the second end of the second water supply tube is positioned external to the second container.

Alternatively or additionally to any of the examples above, the flow control valve can include a button member that moves the flow control valve into the open position while pressed and a spring member that biases the flow control valve to remain in the closed position until the button member is pressed and moves the flow control valve into the closed position after the button member is released from being pressed.

Alternatively or additionally to any of the examples above, the flow control valve can include a valve body and a plug member mechanically coupled to the button member and the spring member. When the button member is pressed, the plug member is unseated from the valve body, and when the button member is released, the plug member is seated against the valve body with biasing force from the spring member.

Alternatively or additionally to any of the examples above, the first container can include a collapsible bag.

Alternatively or additionally to any of the examples above, the first container can include a rigid bottle.

Alternatively or additionally to any of the examples above, the second container can include a collapsible bag.

Alternatively or additionally to any of the examples above, the second container can include a rigid bottle.

Alternatively or additionally to any of the examples above, the container and tube set can further include a first gas supply tube including a first end, a second end, and a third lumen extending therethrough. The third lumen is in operative fluid communication with the second container and the second end of the first gas supply tube is positioned external to the second container.

Alternatively or additionally to any of the examples above, the container and tube set can further include a second gas supply tube including a first end, a second end, and a fourth lumen extending therethrough. The fourth lumen is in operative fluid communication with the second container and the second end of the second gas supply tube is positioned external to the second container.

Alternatively or additionally to any of the examples above, the first end of the second water supply tube can be fluidly coupled to a bottom portion of the second container.

Alternatively or additionally to any of the examples above, the first end of the first gas supply tube can be fluidly coupled to a bottom portion of the second container.

As another example, an endoscopic medical device includes an endoscopic probe and a container and tube set for use in an endoscopic procedure. The container and tube set can be from any of the examples above.

Alternatively or additionally to any of the examples above, the endoscopic medical device can further include a pump in operative fluid communication with the first fluid outlet, the pump configured to receive fluid from the first container and deliver the received fluid to irrigation tubing in the endoscopic probe.

Alternatively or additionally to any of the examples above, the pump can be a peristaltic pump.

Alternatively or additionally to any of the examples above, the container and tube set can be further configured to deliver fluid from the second container to lens wash tubing in the endoscopic probe.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various exemplary embodiments and together with the description serve to explain the principles of the present disclosure.

FIG. 1 depicts components of an endoscope;

FIG. 2 depicts components of an endoscope system with endoscope, light source, light source connector, water reservoir, and tubing assembly for air and lens wash fluid delivery;

FIG. 3A depicts an endoscope system with endoscope, light source, water reservoir, and tubing assembly for hybrid air, lens wash and irrigation fluid delivery, wherein the system is activated to deliver air to atmosphere;

FIG. 3B depicts the endoscope system of FIG. 3A, wherein the system is activated to deliver air to a patient through the patient end of the endoscope;

FIG. 3C depicts the endoscope system of FIG. 3A, wherein the system is activated to deliver lens wash fluid through the patient end of the endoscope;

FIG. 3D depicts the endoscope system of FIG. 3A, wherein the system is activated to deliver irrigation fluid through the patient end of the endoscope;

FIG. 4 depicts a hybrid endoscope system including a video processing unit, connector portion, peristaltic irrigation pump, water reservoir and top, coaxial gas and lens wash supply tubing, upstream and downstream irrigation supply tubing, and alternative gas supply tubing;

FIG. 5 depicts another illustrative endoscope system having an alternative irrigation fluid supply container;

FIG. 6 depicts another illustrative endoscope system having an alternative fluid supply system;

FIGS. 7A-7C depict a button valve for use with a container and tube set; and

FIGS. 8A-8C depict a button-controlled plug for use with a container and tube set.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an exemplary medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, the same or similar reference numbers will be used through the drawings to refer to the same or like parts.

The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” Further, as used herein, the terms “about,” “approximately” and “substantially” indicate a range of values within +/−10% of a stated or implied value. Additionally, terms that indicate the geometric shape of a component/surface refer to exact and approximate shapes.

Embodiments of the present disclosure are described with specific reference to a bottle (e.g., container, reservoir, or the like) and tube assembly or set. It should be appreciated that such embodiments may be used to supply fluid and/or gas to an endoscope, for a variety of different purposes, including, for example to facilitate insufflation of a patient, lens washing, and/or to irrigate a working channel to aid in flushing/suctioning debris during an endoscopic procedure.

Although the present disclosure includes descriptions of a container and tube set suitable for use with an endoscope system to supply fluid and/or gas to an endoscope, the devices, systems, and methods herein could be implemented in other medical systems requiring fluid and/or gas delivery, and for various other purposes.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Conventionally, endoscope devices have been widely used for performing diagnostic and/or therapeutic treatments. During endoscopic procedures, physicians may use a combination of air, irrigation and lens wash as a means of flushing debris, cleaning optics, and insufflating the working lumen. Some systems may use two separate water bottles for irrigation and lens wash while other systems may use a single water bottle for both irrigation and lens wash. As clinicians work through each case, some volume of water is depleted from the water bottle and bottles may need to be replaced one or more times over the course of the day. The process of exchanging bottles may require the user to bend or stoop down, remove the cap and associated inlet tubes from the empty bottle, and place them into a full bottle of sterile water without touching/contaminating the tubes against the external bottle or other non-sterile surfaces (e.g., so as not to create an infection risk to the patient). This may be especially difficult in the single bottle devices where multiple inlet hoses dangle from the cap when removing the cap to replace the sterile water bottle.

Additionally, having the sterile water bottles stowed on lower shelves of the carts, alongside the peristaltic pump, and other equipment may make these difficult to visualize and often, the clinician may not realize that the bottle is nearing empty until they are no longer able to deliver irrigation or lens wash to through the distal end of the scope. There is also an inherent risk associated with stowing the water bottles adjacent to the endoscope control boxes. For example, if the water bottle fails in some way (e.g., leak, burst, rupture, etc.) there may be a high risk of water running or spraying onto these high-cost control systems resulting in significant damage or destruction. Disclosed herein are containers and tube sets that are easily viewed by the clinician and reduces the risk of contamination to the tubes when the container is replaced.

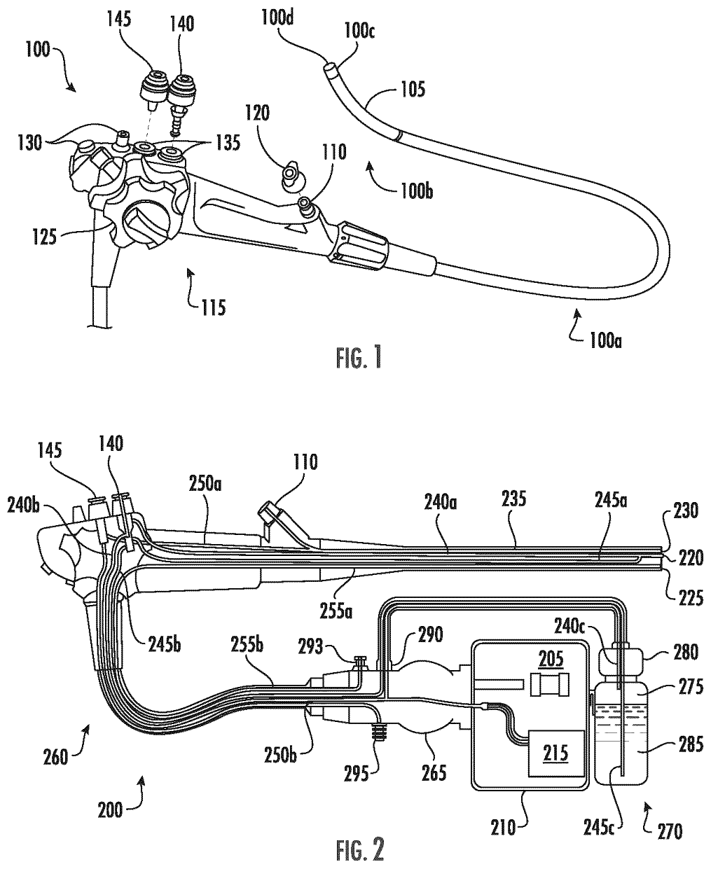

With reference to FIGS. 1-2, an exemplary endoscope 100 and system 200 are depicted that may comprise an elongated shaft 100a that is inserted into a patient. A light source 205 feeds illumination light to a distal portion 100b of the endoscope 100, which may house an imager (e.g., CCD or CMOS imager) (not shown). The light source 205 (e.g., lamp) is housed in a video processing unit 210 that processes signals that are input from the imager and outputs processed video signals to a video monitor (not shown) for viewing. The video processing unit 210 also serves as a component of an air/water feed circuit by housing a pressurizing pump 215, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 extends along the shaft 100a to a proximal channel opening 110 positioned distal to an operating handle 115 of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2). The other valve well 135 receives a suction valve 145 for operating a suction operation. A suction supply line 250a runs distally from the suction valve 145 along the shaft 100a to a junction point in fluid communication with the working channel 235 of the endoscope 100.

The operating handle 115 is electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) is fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The detachable gas/lens wash connection 290 may be detachable from the connector portion 265 and/or the gas supply tubing 240c. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, passes through the top 280 of the reservoir 270 to the same detachable connection 290 as the gas supply tubing 240c on the connector portion 265. In other embodiments, the connections may be separate and/or separated from each other. The connector portion 265 also has a detachable irrigation connection 293 for irrigation supply tubing (not shown) running from a source of irrigation water (not shown) to the irrigation feed line 255b in the umbilical 260. The detachable irrigation connection 293 may be detachable from the connector portion 265 and/or the irrigation supply tubing (not shown). In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100. The detachable suction connection 295 may be detachable from the connector portion 265 and/or the suction feed line 250b and/or the vacuum source.

The gas feed line 240b and lens wash feed line 245b are fluidly connected to the valve well 135 for the gas/water valve 140 and configured such that operation of the gas/water valve 140 in the well controls supply of gas or lens wash to the distal tip 100c of the endoscope 100. The suction feed line 250b is fluidly connected to the valve well 135 for the suction valve 145 and configured such that operation of the suction valve in the well controls suction applied to the working channel 235 of the endoscope 100.

Referring to FIG. 2, an exemplary operation of an endoscopic system 200, including an endoscope such as endoscope 100 above, is explained. Air from the air pump 215 in the video processing unit 210 is flowed through the connector portion 265 and branched to the gas/water valve 140 on the operating handle 115 through the gas feed line 240b in the umbilical 260, as well as through the gas supply tubing 240c to the water reservoir 270 via the connection 290 on the connector portion 265. When the gas/water valve 140 is in a neutral position, without the user's finger on the valve, air is allowed to flow out of the valve to atmosphere. In a first position, the user's finger is used to block the vent to atmosphere. Gas is allowed to flow from the valve 140 down the gas supply line 240a and out the distal tip 100c of the endoscope 100 in order to, for example, insufflate the treatment area of the patient. When the gas/water valve 140 is pressed downward to a second position, gas is blocked from exiting the valve, allowing pressure of the air passing from the air pump 215 to rise in the water reservoir 270. Pressurizing the water source forces water out of the lens wash tubing 245c, through the connector portion 265, umbilical 260, through the gas/water valve 140 and down the lens wash supply line 245a, converging with the gas supply line 240a prior to exiting the distal tip 100c of the endoscope 100 via the gas/lens wash nozzle 220. Air pump pressure may be calibrated to provide lens wash water at a relatively low flow rate compared to the supply of irrigation water.

The volume of the flow rate of the lens wash is governed by gas pressure in the water reservoir 270. When gas pressure begins to drop in the water reservoir 270, as water is pushed out of the reservoir 270 through the lens wash tubing 245c, the air pump 215 replaces lost air supply in the reservoir 270 to maintain a substantially constant pressure, which in turn provides for a substantially constant lens wash flow rate. In some embodiments, a filter (not shown) may be placed in the path of the gas supply tubing 240c to filter-out undesired contaminants or particulates from passing into the water reservoir 270. In some embodiments, outflow check valves or other one-way valve configurations (not shown) may be placed in the path of the lens wash supply tubing to help prevent water from back-flowing into the reservoir 270 after the water has passed the valve.

A relatively higher flow rate of irrigation water is typically required compared to lens wash, since a primary use is to clear the treatment area in the patient of debris that obstructs the user's field of view. Irrigation is typically achieved with the use of a pump (e.g., peristaltic pump), as described. In embodiments with an independent water source for irrigation, tubing placed in the bottom of a water source is passed through the top of the water source and threaded through the head on the upstream side of the pump. Tubing on the downstream side of the pump is connected to the irrigation feed line 255b in the umbilical 260 and the irrigation supply line 255a endoscope 100 via the irrigation connection 293 on the connector portion 265. When irrigation water is required, fluid is pumped from the water source by operating the irrigation pump, such as by depressing a footswitch (not shown), and flows through the irrigation connection 293, through the irrigation feed line 255b in the umbilical, and down the irrigation supply line in the shaft 100a of the endoscope to the distal tip 100c. In order to equalize the pressure in the water source as water is pumped out of the irrigation supply tubing, an air vent (not shown) may be included in the top 280 of the water reservoir 270. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown), similar to the lens wash tubing 245c, may be placed in the path of the irrigation supply tubing to help prevent back-flow into the reservoir after water has passed the valve.

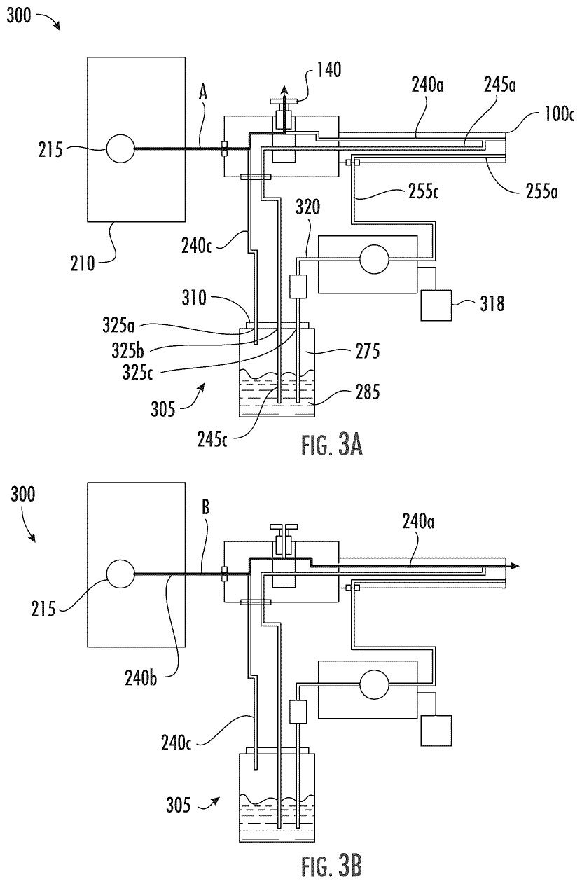

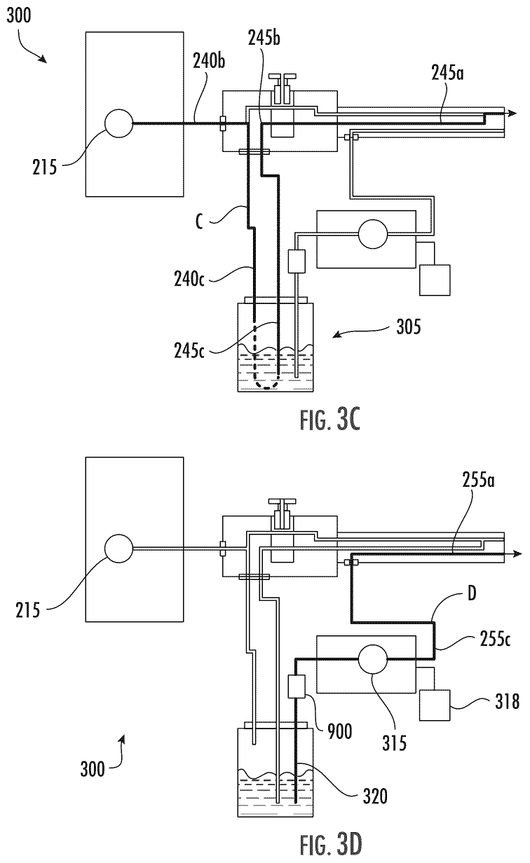

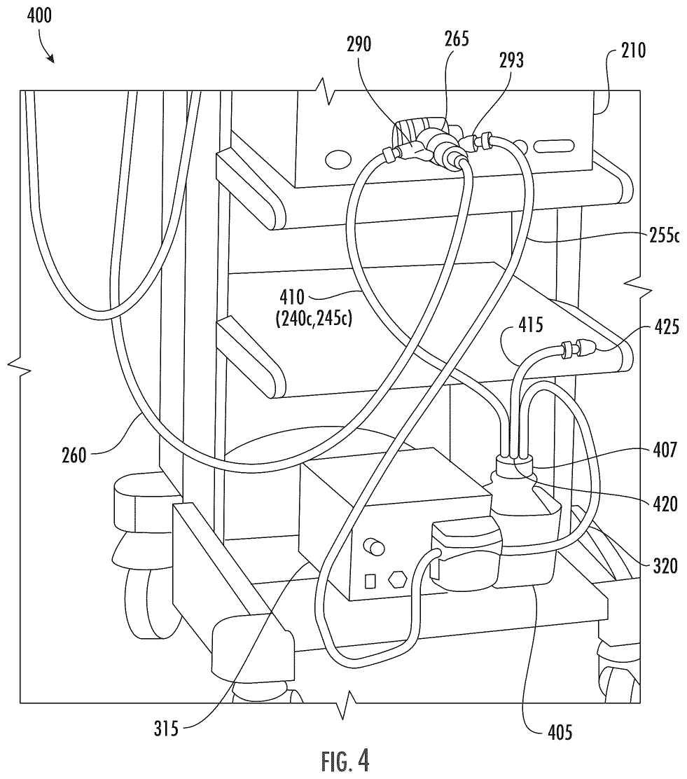

FIGS. 3A-3D are schematic drawings illustrating the operation of an embodiment of a hybrid system 300 where the supply tubing for irrigation and lens wash are connected to and drawn from a single water reservoir. It is contemplated that fluids other than water may be used, such as, but not limited to saline. The hybrid system 300 includes the single water reservoir 305, a cap 310 for the reservoir, gas supply tubing 240c, lens wash supply tubing 245c, irrigation pump 315 with foot switch 318, upstream supply tubing for irrigation 320 and downstream irrigation supply tubing 255c. The cap 310 may be configured to attach in a seal-tight manner to the water reservoir 305 by a typically threaded arrangement. The cap 310 may include a gasket to seal the cap 310 to the reservoir 305. The gasket can be an O-ring, flange, collar, and/or the like and can be formed of any suitable material. A number of through-openings (325a, 325b, 325c) in the cap 310 are provided to receive, respectively, the gas supply tubing 240c, lens wash supply tubing 245c, and upstream irrigation supply tubing 320. In FIGS. 3A-3D, the system depicted includes separate tubing for gas supply, lens wash, and irrigation.

In other embodiments, the gas supply tubing 240c and lens wash tubing 245c may be combined in a coaxial arrangement. Some illustrative coaxial arrangements are described in commonly assigned U.S. patent application Ser. No. 17/558,239, titled INTEGRATED CONTAINER AND TUBE SET FOR FLUID DELIVERY WITH AN ENDOSCOPE and U.S. patent application Ser. No. 17/558,256, titled TUBING ASSEMBLIES AND METHODS FOR FLUID DELIVERY, the disclosures of which are hereby incorporated by reference. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the water reservoir (see, e.g., gas and lens wash supply tubing 240c, 245c). The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion (e.g., connector portion 265 of FIG. 2).

In various embodiments, different configurations of valving (not shown) may be incorporated into various embodiments disclosed hereby, including the tubing of the system 200, 300. For example, an in-flow check valve can be disposed in the path of the gas supply tubing 240c to help prevent backflow into the air pump 215. In this manner, pressure building within the water reservoir 305 creates a pressure difference between the water source and the gas supply tubing 240c helping to maintain a positive pressure in the water source even when large amounts of water may be removed from the water source during the irrigation function. This arrangement compensates for any time lag in air being delivered from the air pump 215 to the water reservoir 305, which might otherwise cause a negative pressure vacuum in the water reservoir. Similarly, an out-flow check valve, such as the one-way valve with inlet/outlets and valve insert, may be incorporated in the lens wash supply tubing 245c, upstream irrigation supply tubing 320, and/or downstream irrigation supply tubing 255c to help prevent backflow of water from either or both of the lens wash and supply tubing for irrigation in the event of a negative pressure situation, as described.

More generally, in many embodiments, a check valve may refer to any type of configuration for fluid to flow only in one direction in a passive manner. For example, a check valve may include, or refer to, one or more of a ball check valve, a diaphragm check valve, a swing check valve, a tilting disc check valve, a flapper valve, a stop-check valve, a lift-check valve, an in-line check valve, a duckbill valve, a pneumatic non-return valve, a reed valve, a flow check. Accordingly, a check valve as used herein is meant to be separate and distinct from an active valve that is operated in a binary manner as an on/off valve or switch to allowed flow to be turned on or allow flow to be turned off (e.g., a stop cock valve, solenoid valve, peristaltic pump).

During operation of the system of FIGS. 3A-3D, a flow of water for irrigation may be achieved by operating the irrigation pump 315. A flow of water for lens wash may be achieved by depressing the gas/water valve 140 on the operating handle 115 of the endoscope 100. These functions may be performed independent of one another or simultaneously. When operating lens wash and irrigation at the same time, as fluid is removed from the water reservoir 305, the pressure in the system may be controlled to maintain the lens wash supply tubing 245c at substantially the pressure necessary to accomplish a lower flow rate lens wash, while compensating for reduced pressure in the water reservoir 305 due to supplying a high flow rate irrigation. When pressure is reduced in the water reservoir by use of the lens wash function, the irrigation function, or both functions simultaneously, the reduced pressure may be compensated for by the air pump 215 via the gas supply tubing 240c.

The schematic set-up in FIGS. 3A-3D has been highlighted to show the different flow paths possible with the hybrid system 300 having supply tubing for irrigation 320 and lens wash supply tubing 245c connected to and drawn from the single water reservoir 305. As shown in FIG. 3A, the endoscope 100 is in a neutral state with the gas/water valve 140 in an open position. The neutral state delivers neither gas, nor lens wash, to the distal tip of the endoscope. Rather gas (pressure) is delivered along path A from the pressurizing air pump 215 and vented through the gas feed line 240b in the umbilical 260 via the connector portion 265 and through the gas/water valve to atmosphere. Since the system is open at the vent hole in the gas/water valve 140, there is no build up to pressurize the water reservoir 305 and consequently no water is pushed through the lens wash supply tubing 245c.

As shown in FIG. 3B, the endoscope 100 is in a gas delivery state with the gas/water valve 140 in a first position. When gas is called for at the distal tip 100c, for example, to clean the end face 100d of the distal tip or insufflate the patient body in the treatment area, the user closes off the vent hole in the gas/water valve 140 with a thumb, finger, or the like (first position). In this state, gas (pressure) is delivered along path B from the air pump 215 and flowed through the gas feed line 240b in the umbilical 260 via the connector portion 265. The gas continues through the gas/water valve 140 to the gas supply line 240a in the endoscope shaft 100a and out the gas/lens wash nozzle 220 at the distal tip 100c. There is no build up to pressurize the water reservoir since the system is open at the gas/lens water nozzle 220, and consequently no water is pushed through the lens wash supply tubing 245c.

As shown in FIG. 3C, the endoscope 100 is in a lens wash delivery state with the gas/water valve 140 in a second position. When lens wash is called for at the distal tip 100c, for example, to clean the end face 100d of the distal tip 100c, the user, keeping the vent hole in the air/water valve closed off, depresses the valve 140 to its furthest point in the valve well 135. The second position blocks off the gas supply to both atmosphere and the gas supply line 240a in the endoscope and opens up the gas/water valve 140 to allow lens wash water to pass through to the lens wash supply line 245a in the endoscope shaft 100a and out the gas/lens wash nozzle 220 at the distal tip 100c. In this state, gas (pressure) is delivered along path C from the air pump 215, through the branched line in the connector portion 265 and out of the gas supply tubing 240c to the water reservoir 305. The gas (pressure) pressurizes the surface of the remaining water 285 in the reservoir 305 and pushes water up the lens wash supply tube 245c to the connector portion 265. The pressurized lens wash water is pushed further through the lens wash feed line 245b in the umbilical 260 and through the gas/water valve 140. Since the system 300 is closed, gas pressure is allowed to build and maintain a calibrated pressure level in the water reservoir 305, rather than venting to atmosphere or being delivered to the patient. This pressure, along with the endoscope feed and supply lines and external tubing, translates to a certain range of flow rate of the lens wash.

As shown in FIG. 3D, the endoscope 100 is in an irrigation delivery state. This may be performed at the same or a different time from the delivery of gas and/or lens wash. When irrigation is called for at the distal tip 100c, for example, if visibility in the treatment area is poor or blocked by debris, or the like, the user activates the irrigation pump 315 (e.g., by depressing foot switch 318) to deliver water along path D. With the pump 315 activated, water is sucked out of the water reservoir 305 through the upstream irrigation supply tubing 320 and pumped along the downstream irrigation supply tubing 255c to the connector portion 265. The irrigation pump head pressure pushes the irrigation water further through the irrigation feed line 255b in the umbilical 260, through the irrigation supply line 255a in the endoscope shaft 100a, and out the irrigation opening 225 at the distal tip 100c. The irrigation pump pressure may be calibrated, along with the endoscope irrigation feed and supply lines and external tubing, to deliver a certain range of flow rate of the irrigation fluid.

FIG. 4 is a schematic drawing illustrating a further embodiment of a hybrid system 400 including a video processing unit 210, connector portion 265, peristaltic irrigation pump 315, water reservoir 405 and top 407, coaxial gas and lens wash supply tubing 410, upstream and downstream irrigation supply tubing 320, 255c, respectively, and alternative gas (e.g., CO₂) supply tubing 415. A length of the alternative gas supply tubing 415 passes from one end positioned in the gas gap 275 (see FIG. 2) between the top 407 of the water reservoir 405 and the remaining water 285 in the reservoir through an additional opening 420 in the top of the reservoir to a detachable connection 425 for a source of the alternative gas supply (e.g., CO₂ hospital house gas source). When the alternative gas supply is desired, such as CO₂ gas, the air pump 215 on the video processing unit 210 may be turned off and CO₂ gas, rather than air, is thereby flowed to the water reservoir 405 pressurizing the water surface. Generally, the flow of CO₂ through the endoscope 100 is similar to the flow of air. In the neutral state, CO₂ gas flows backward up the gas supply tubing 240c to the connector portion 265, up the gas feed line 240b, and is vented through the gas/water valve 140 to atmosphere. In the first position, the user closes off the vent hole in the gas/water valve 140, and the CO₂ gas is flowed through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100 and opens up the gas/water valve 140 to allow lens wash water to pass through to the lens wash supply line 245a in the endoscope shaft 100a and out the gas/lens wash nozzle 220 at the distal tip 100c. Gas (pressure) in the reservoir 405 is maintained by delivering gas through alternative gas (e.g., CO₂) supply tubing 415. The irrigation function may be accomplished in a similar manner to the operation described above with respect to FIG. 3D.

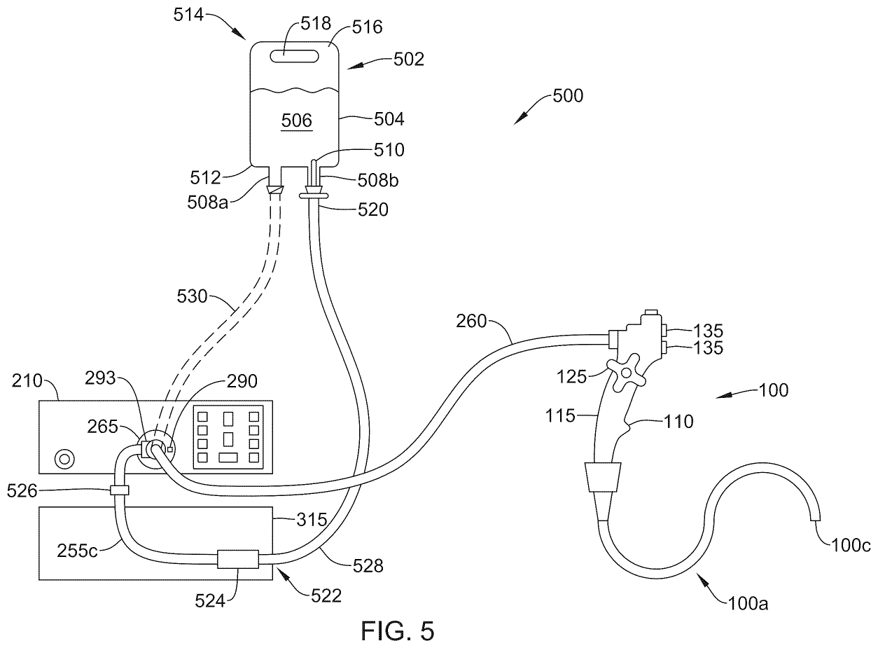

As described above, it may be desirable to reduce opportunities for contamination to the tube set 240c, 245c, 320, 410, 415 during replacement of the water reservoir(s). FIG. 5 depicts a schematic view of an illustrative endoscopic system 500 which may reduce the number of water reservoir changes and/or reduce opportunities for contamination during replacement of the water reservoir(s). The system 500 may include components similar to the endoscope and endoscope systems described with regard to FIGS. 1-4; however, not all features may be described or shown here.

The system 500 of FIG. 5 depicts an illustrative fluid reservoir 502 that may include a number of advantages over the current bottle system described above. The reservoir 502 may include a container 504 configured to hold a fluid 506. In the illustrated embodiment, the container 504 is fluidly coupled to the upstream irrigation tubing 528 and is configured to provide fluid for irrigation to the endoscope 100. Generally, the irrigation tubing 528 may be a water or fluid supply line or tube for supplying water or other fluid to an endoscope 100. Alternatively, or additionally, the container 504 may be coupled to a gas and lens wash supply tubing 530 to provide insufflation and fluid for clearing the lens. Further, in some cases, the reservoir 502 may supply fluid for both irrigation and lens wash. While not explicitly shown, the reservoir 502 may include printed lines, numbers, or other visual indicia to allow a user to easily determine how much fluid is left in the reservoir 502.

The container 504 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the container 504 may be entirely translucent, entirely opaque, or combinations thereof. In some cases, the container 504 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the container 504 may be variable. For example, the volume of the container 504 may be 500 milliliters (mL) or greater, 1000 mL or greater, 2000 mL or greater, 3000 mL, 4000 mL or greater, etc. The volume may be less than 500 mL or greater than 4000 mL, as desired. The reservoir 502 may be pre-filled (e.g., prior to entering the procedure suite or at the time of manufacturing) with water or other fluid. In some cases, the clinician may select the reservoir 502 from a plurality of differently sized available reservoirs based on the number and/or types of procedures expected for a day. By selecting a reservoir 502 having a volume large enough to accommodate an entire day of procedures, the need for replacing the sterile fluid source (e.g., the reservoir 502) may be reduced or eliminated. It is contemplated that flexible bags may utilize less plastic (or other material) than a bottle designed to hold a similar amount of fluid. Thus, the use of a flexible bag as a fluid reservoir 502 may increase the level of environmental sustainability of the system 500. For example, if the user sets up the system with a 3000 mL (3 liter) bag reservoir 502 and therefore does not need to utilize three individual one-liter bottles, a significant reduction of waste may be realized. It is further contemplated that when disposed of or discarded, a bag reservoir 502 may occupy less volume than a bottle capable of holding an equivalent amount of fluid.

The reservoir 502 may further include one or more ports 508a, 508b, such as, but not limited to a spike port or a septum port, extending from and in selective fluid communication with an interior of the container 504. The ports 508a, 508b may be formed as a monolithic structure with the first container 504. The ports 508a, 508b may be generally tubular structures with each port 508a, 508b defining a lumen extending therethrough. The lumens of the ports 508a, 508b may be configured to selectively fluidly couple the interior of the container 504 with another component, such as, but not limited to, a water or fluid supply tube. In some embodiments, the ports 508a, 508b may be positioned adjacent to a bottom end 512 of the reservoir 502. However, this is not required. The ports 508a, 508b may be positioned in other locations, as desired. If the ports 508a, 508b are positioned at a location other than the bottom end 512 of the container 504, a dip tube or tube extension may be required to access the fluid at the bottom of the container 504. In some cases, at least one port 508b may be configured to be coupled to the upstream irrigation tubing (or water/fluid supply tube) 528 while the other port 508a may be configured to allow the user to add additives to the fluid 506 (e.g., irrigation fluid). While the reservoir 502 is illustrated as including two ports 508a, 508b, the reservoir 502 may include one port or more than two ports, as desired.

While not explicitly shown, the ports 508a, 508b may each include a removable cap or seal configured to form a fluid tight seal with the port 508a, 508b. The removable cap or seal may help to maintain the sterility of the ports 508a, 508b. The removable cap or seal may be coupled to a free end of the ports 508a, 508b using a number of different techniques. For example, the cap or seal may be coupled to the port 508a, 508b using a threaded engagement, a friction fit, a snap fit, etc. In other instances, the cap or seal may be removed through a twisting motion configured to break the cap or seal from the port 508a, 508b. Once the cap or seal has been removed, the port 508a, 508b may be pierced with a spike tip or spike port adaptor 510 that is coupled to the upstream irrigation tubing 528. For example, in addition to the removable cap or seal, the port 508a, 508b may include an internal seal disposed within a lumen of the port 508a, 508b that may be punctured or pierced by the spike port adaptor 510. The internal seal may be configured to prevent fluid 506 from leaking from the container 504 prior to the spike port adaptor 510 being inserted into the port 508a, 508b. In some embodiments, the internal seal may be self-sealing such that upon removal of the spike port adaptor 510 fluid is prevented from leaking from the port 508a, 508b. The outer surface of the spike port adaptor 510 may form an interference fit with the inner surface of the port 508a, 508b. The fit and/or coupling between the spike port adaptor 510 and the port 508a, 508b may be sufficient to remain in place when the irrigation supply tube 528 and/or other tubing sets are coupled to the spike port adaptor 510. It is contemplated that the spike port adaptor 510 may be inserted into one of the ports 508a, 508b utilizing universally used aseptic techniques such as those used with IV fluid bags. This may help reduce infection risk by maintaining sterile components, not introducing contaminants into the fluid 506, etc. It is further contemplated that additives may be added to the fluid 506 using similar aseptic techniques via one of the ports 508a, 508b.

The reservoir 502 may include a handle 516 positioned adjacent to a top portion 514 thereof. The handle 516 may define an opening or through hole 518 for receiving a hand or hook therethrough to carry the reservoir 502. In some cases, the handle 516 may include an undulating surface configured to provide a more ergonomic grip for the user. It is contemplated that the handle 516 may be formed from a similar material as the container 504 or a different material, as desired. In some examples, the handle 516 may be formed from polyethylene terephthalate (PET), polypropylene (PP), etc. The handle 516 may allow the reservoir 502 to be hung from a hook, such as, but not limited to an IV stand. Hanging the reservoir 502 may allow the reservoir 502 to be positioned above the level of an endoscope cart which may enable the user to see the fluid 506 level at any time. This may help the clinician avoid running out of fluid during a procedure. Additionally, elevating the reservoir may eliminate the need for the clinician to bend or stoop during setup of the system 500 and/or to change the reservoir 502. In some cases, head pressure generated from the elevating the reservoir 502 may enable rapid priming of the irrigation circuit (and/or lens wash circuit if so connected) which may save time during setup. It is further contemplated that hanging the reservoir 502 from a hook or IV stand may allow the reservoir 502 to be positioned away from expensive capital equipment thus reducing or eliminating the potential for fluid running or flowing inadvertently onto the capital equipment and causing damage or destruction.

The reservoir 502 may be connected in fluid communication with a lumen of an upstream irrigation supply tube 528. The upstream irrigation supply tube 528 extends from a second end region 522 external to the container 504 and positioned within a pump head 524 of the peristaltic irrigation pump 315 to a first end 520. The first end 520 is coupled to the spike port adaptor 510 which in turn is configured to extend through a lumen of the port 508b and pierce a seal within the lumen of the port 508b to fluidly couple the interior of the container 504 with the lumen of the upstream irrigation supply tube 528. The second end of the upstream irrigation supply tube 528 is configured to be fluidly coupled with an irrigation lumen of the endoscope 100. When irrigation water is required, fluid is pumped from the container 504 by operating the irrigation pump 315, such as by depressing a footswitch (not shown), and flows from the reservoir 502, through the upstream irrigation supply tubing 528, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 526 positioned in line with the downstream irrigation supply tubing 255c. The flow control valve 526 may prevent the unintentional flow of fluid from the container 504 to the endoscope 100. In some cases, the flow control valve 526 may be configured to open when the pressure within the downstream irrigation supply line 255c reaches a predetermined minimum pressure. It is contemplated that the predetermined minimum pressure may be greater than the head pressure created by the height differential between the reservoir 502 and the irrigation pump 315. The flow control valve 526 may also prevent fluid from leaking from the downstream irrigation supply tube 255c when the endoscope 100 is changed between patients.

In some embodiments, the irrigation pump 315 may be omitted. For example, the reservoir 502 may be inserted into a compression sleeve. When irrigation fluid is desired, the compression sleeve may be activated to exert pressure on an outer surface of the reservoir 502 and to provide the required pressure to perform irrigation at the distal end of the endoscope 100.

In some embodiments, a lens wash supply tubing 530 may be coupled to the first port 508a. In this embodiment, the lens wash supply tubing and the irrigation supply tubing 528 may be coupled to the same reservoir 502. The reservoir 502 may be inserted into a compression sleeve, as described above. The pressure exerted by the compression sleeve may be sufficient to supply pressurized fluid for both irrigation and lens wash to the endoscope without the use of a secondary pump (such as an irrigation pump) or pressure source (such as an insufflator).

If there is a need to replace the reservoir 502 with a new full bag, for example when the reservoir 502 is empty or near empty, the user may, optionally, hang the new bag near the reservoir 502 to be replaced. The user may then disengage the spike port adaptor 510 from the port 508b and insert the spike port adaptor 510 into a port of the new bag. This may be performed without requiring the clinician to bend or stoop to access the reservoir 502. The port 508b may self-seal to prevent fluid leaks from the reservoir 502 as it is being replaced or upon removal of the spike port adaptor 510. This method of replacing the reservoir 502 may have a lower risk of introducing contaminants into the systems relative to traditional bottle systems. For example, the change out method described herein may allow the reservoir 502 to be changed out without having tubing dangling from a cap (as in a bottle system). Further, the system 500 may remain largely closed as the reservoir 502 is changed out.

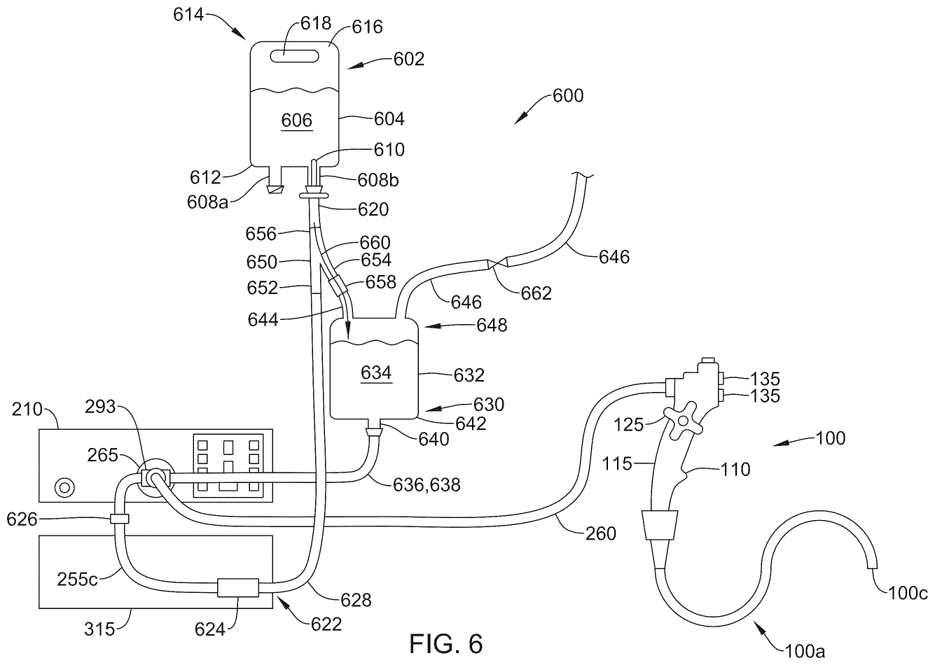

FIG. 6 depicts a schematic view of another illustrative endoscopic system 600 which may reduce the number of water reservoir changes and/or reduce opportunities for contamination during replacement of the water reservoir(s). The system 600 may include a number of advantages over the current bottle system described above. The system 600 may include components similar to the endoscope and endoscope systems described with regard to FIGS. 1-4; however, not all features may be described or shown here.

Generally, the system 600 may include a first reservoir 602 and a second reservoir 630. The first reservoir 602 may be configured to supply water or fluid for both irrigation (e.g., via the first reservoir 602) and lens wash (e.g., via the second reservoir 630). This may allow a single fluid source to be used to provide fluid for both irrigation and lens wash. While not explicitly shown, the reservoirs 602, 630 may include printed lines, numbers, or other visual indicia to allow a user to easily determine how much fluid is left in the reservoirs 602, 630.

The first reservoir 602 may include a first container 604 configured to hold a first volume of fluid 606. In the illustrated embodiment, the first container 604 is fluidly coupled to the upstream irrigation supply tubing 628 and is configured to provide fluid for irrigation to the endoscope 100. Generally, the irrigation supply tubing 628 may be a water or fluid supply line or tube for supplying water or other fluid to an endoscope. Additionally, the first container 604 may be selectively fluidly coupled to a second fluid reservoir 630. The second reservoir 630 may include a second container 632 configured to hold a second volume of fluid 634. In the illustrated embodiment, the second container 632 is fluidly coupled to the gas and lens wash supply tubing 636, 638 and is configured to provide fluid for lens wash to the endoscope 100. Generally, the lens wash supply tubing 638 may be a water or fluid supply line or tube for supplying water or other fluid to an endoscope. The gas and lens wash supply tubing 636, 638 may be coaxially arranged. For example, the gas supply tubing may define a lumen that is sufficiently large in diameter to encompass a smaller diameter lens wash tubing, coaxially received within the gas supply tubing, as well as provide air to the water source in an annular space surrounding the lens wash tubing to pressurize the second reservoir 630. The lens wash supply tubing may be configured to exit the lumen defined by the coaxial gas supply tubing in any suitable sealed manner, such as, for example, an aperture, fitting, collar, and/or the like, for the purpose of transitioning from the coaxial arrangement to a side-by-side arrangement at the detachable gas/lens wash connection to the endoscope connector portion 265. In other embodiments, the gas and lens wash supply tubing 636, 638 may be arranged in a side-by-side arrangement.

The first and second containers 604, 632 may be formed from one or more layers of a lightweight, flexible material, such as, but not limited to, low density polyethylene (LDPE), thermoplastic polyurethane (TPU), silicone, polyethylene terephthalate (PET), aluminum, nylon, polyethylene (PE), plasticized polyvinyl chloride (PVC), or combinations thereof, etc. In some embodiments, the first and second containers 604, 632 may be entirely translucent, entirely opaque, or combinations thereof. In some cases, the first and second containers 604, 632 may be a flexible bag analogous to those utilized to deliver intravenous replacement fluid in clinical settings (for example, an intravenous (IV) fluid bag). Such bags may be readily available and familiar to the clinician as they are widely used in various sizes. The volume of the first and second containers 604, 632 may be variable. For example, the volume of the first container 604 and/or the second container 632 may be 500 milliliters (mL) or greater, 1000 mL or greater, 2000 mL or greater, 3000 mL, 4000 mL or greater, etc. The volume may be less than 500 mL or greater than 4000 mL, as desired. One or both of the first and second reservoirs 602, 630 may be pre-filled (e.g., prior to entering the procedure suite or at the time of manufacturing) with water or other fluid. In some cases, the clinician may select the reservoir(s) 602, 630 from a plurality of differently sized available reservoirs based on the number and/or types of procedures expected for a typical or the specific day. In the illustrated embodiments, the first reservoir 602 may supply fluid to the second reservoir 630. By selecting a first reservoir 602 having a volume large enough to accommodate an entire day of procedures, the need for replacing the sterile fluid source (e.g., the first reservoir 602) may be reduced or eliminated. In some cases, the first reservoir 602 may be used to periodically refill the second reservoir 630. Thus, the volume of the first reservoir 602 may be greater than the volume of the second reservoir 630, although this is not required. It is further contemplated that, in some embodiments, one or both of the first or second reservoirs 602, 630 may be a rigid bottle.

It is contemplated that flexible bags may utilize less plastic (or other material) than a bottle designed to hold a similar amount of fluid. Thus, the use of a flexible bag as a fluid reservoir 602, 630 may increase the level of environmental sustainability of the system 600. For example, if the user sets up the system with a 3000 mL (3 liter) bag reservoir 602 and therefore does not need to utilize three individual one liter bottles, a significant reduction of waste may be realized. It is further contemplated that when disposed of or discarded, a flexible bag reservoir may occupy less volume than a bottle capable of holding an equivalent amount of fluid.

The first reservoir 602 may further include one or more ports 608a, 608b, such as, but not limited to a spike port or a septum port, extending from and in selective fluid communication with an interior of the first container 604. The ports 608a, 608b may be formed as a monolithic structure with the first container 604. The ports 608a, 608b may be generally tubular structures with each port 608a, 608b defining a lumen extending therethrough. The lumens of the ports 608a, 608b may be configured to selectively fluidly couple the interior of the first container 604 with another component, such as, but not limited to, a fluid or water supply tube. In some embodiments, the ports 608a, 608b may be positioned adjacent to a bottom end 612 of the first reservoir 602. However, this is not required. The ports 608a, 608b may be positioned in other locations, as desired. If the ports 608a, 608b are positioned at a location other than the bottom end 612 of the first container 604, a dip tube or tube extension may be required to access the fluid at the bottom of the first container 604. In some cases, at least one port 608b may be configured to be coupled to the upstream irrigation tubing (or water supply tube) 628 while another port 608a may be configured to allow the user to add additives to the fluid 606. While the first reservoir 602 is illustrated as including two ports 608a, 608b, the first reservoir 602 may include one port or more than two ports, as desired.

While not explicitly shown, the ports 608a, 608b may each include a removable cap or seal configured to form a fluid tight seal with the port 608a, 608b. The removable cap or seal may help to maintain the sterility of the ports 608a, 608b. The removable cap or seal may be coupled to a free end of the ports 608a, 608b using a number of different techniques. For example, the cap or seal may be coupled to the port 608a, 608b using a threaded engagement, a friction fit, a snap fit, etc. In other instances, the cap or seal may be removed through a twisting motion configured to break the cap or seal from the port 608a, 608b. Once the cap or seal has been removed, the port 608a, 608b may be pierced with a spike tip or spike port adaptor 610 that is coupled to the upstream irrigation tubing 628. For example, in addition to the removable cap or seal, the port 608a, 608b may include an internal seal disposed within a lumen of the port 608a, 608b that may be punctured or pierced by the spike port adaptor 610. The internal seal may be configured to prevent fluid 606 from leaking from the first container 604 prior to the spike port adaptor 610 being inserted into the port 608a, 608b. In some embodiments, the internal seal may be self-sealing such that upon removal of the spike port adaptor 610 fluid is prevented from leaking from the port 608a, 608b. The outer surface of the spike port adaptor 610 may form an interference fit with the inner surface of the port 608a, 608b. The fit and/or coupling between the spike port adaptor 610 and the port 608a, 608b may be sufficient to remain in place when the irrigation supply tube 628, branched connector 650, and/or other tubing sets are coupled to the spike port adaptor 610. It is contemplated that the spike port adaptor 610 may be inserted into one of the ports 608a, 608b utilizing universally used aseptic techniques such as those used with IV fluid bags. This may help reduce infection risk by maintaining sterile components, not introducing contaminants into the fluid 606, etc. It is further contemplated that additives may be added to the fluid 606 using similar aseptic techniques via one of the ports 608a, 608b.

The first reservoir 602 may include a handle 616 positioned adjacent to a top portion 614 thereof. The handle 616 may define an opening or through hole 618 for receiving a hand or hook therethrough to carry the first reservoir 602. In some cases, the handle 616 may include an undulating surface configured to provide a more ergonomic grip for the user. It is contemplated that the handle 616 may be formed from a similar material as the first container 604 or a different material, as desired. In some examples, the handle 616 may be formed from polyethylene terephthalate (PET), polypropylene (PP), etc. The handle 616 may allow the first reservoir 602 to be hung from a hook, such as, but not limited to an IV stand. Hanging the first reservoir 602 may allow the first reservoir 602 to be positioned above the level of an endoscope cart which may enable the user to see the fluid 606 level at any time. This may help the clinician avoid running out of fluid during a procedure. Additionally, elevating the reservoir may eliminate the need for the clinician to bend or stoop during setup of the system 600 and/or to change the first reservoir 602. In some cases, head pressure generated from the elevating the first reservoir 602 may enable rapid priming of the irrigation circuit (and/or lens wash circuit if so connected) which may save time during setup. It is further contemplated that hanging the first reservoir 602 from a hook or IV stand may allow the first reservoir 602 to be positioned away from expensive capital equipment thus reducing or eliminating the potential for fluid running or flowing inadvertently onto the capital equipment and causing damage or destruction.

The first reservoir 602 may be connected in fluid communication with a lumen of the upstream irrigation supply tube 628. The upstream irrigation supply tube 628 extends from a second end region 622 external to the container 604 and positioned within a pump head 624 of the peristaltic irrigation pump 315 to a first end 620. The first end 620 of the upstream irrigation supply tube 628 is coupled to the spike port adaptor 610 which in turn is configured to extend through a lumen of the port 608b and pierce a seal within the lumen of the port 608b to fluidly couple the interior of the container 604 with the lumen of the upstream irrigation supply tube 628. The second end of the upstream irrigation supply tube 628 is configured to be fluidly coupled with an irrigation lumen of the endoscope 100. When irrigation water is required, fluid is pumped from the first container 604 by operating the irrigation pump 315, such as by depressing a footswitch (not shown), and flows from the first reservoir 602, through the upstream irrigation supply tubing 628 and a branched connector 650, through the downstream irrigation supply tubing 255c, through the irrigation connection 293, through the irrigation feed line 255b in the umbilical 260, and down the irrigation supply line 255a in the shaft 100a of the endoscope to the distal tip 100c.

The downstream irrigation supply tubing 255c may include a loaded check valve or flow control valve 626 positioned in line with the downstream irrigation supply tubing 255c. The flow control valve 626 may prevent the unintentional flow of fluid from the first container 604 to the endoscope 100. In some cases, the flow control valve 626 may be configured to open when the pressure within the downstream irrigation supply line 255c reaches a predetermined minimum pressure. It is contemplated that the predetermined minimum pressure may be greater than the head pressure created by the height differential between the first reservoir 602 and the irrigation pump 315. The flow control valve 626 may also prevent fluid from leaking from the downstream irrigation supply tube 255c when the endoscope 100 is changed between patients and the tubing set connector is separated from the endoscope water port.

In some embodiments, the irrigation pump 315 may be omitted. For example, the reservoir 602 may be inserted into a compression sleeve. When irrigation fluid is desired, the compression sleeve may be activated to exert pressure on an outer surface of the reservoir 602 and to provide the required pressure to perform irrigation at the distal end of the endoscope 100. In another embodiment the reservoir 602 may be inserted into a compression sleeve which applies constant pressure to the reservoir 602 with a flow switch positioned along irrigation supply tubing 628 to provide binary control of irrigation flow.

The second reservoir 630 may further include one or more ports 640, such as, but not limited to a spike port or a septum port, extending from and in selective fluid communication with an interior of the second container 632. The port 640 may be formed as a monolithic structure with the second container 632. The port 640 may be a generally tubular structure with the port 640 defining a lumen extending therethrough. The lumen of the port 640 may be configured to selectively fluidly couple the interior of the second container 632 with another component, such as, but not limited to, fluid/water/gas supply tube(s). In some cases, the port 640 may be configured to be coupled to the gas and lens wash supply tubing 636, 638. In some embodiments, the port 640 may be positioned adjacent to a bottom end 642 of the second reservoir 630. However, this is not required. The port 640 may be positioned in other locations, as desired. If the port 640 is positioned at a location other than the bottom end 642 of the second container 632, a dip tube or tube extension may be required (e.g., coupled to the lens wash supply tubing 638) to access the fluid at the bottom of the second container 632. While the second reservoir 630 is illustrated as including one port 640, the second reservoir 630 may include more than one port, as desired.

While not explicitly shown, the port 640 may include a removable cap or seal configured to form a fluid tight seal with the port 640. The removable cap or seal may help to maintain the sterility of the port 640. The removable cap or seal may be coupled to a free end of the ports 640 using a number of different techniques. For example, the cap or seal may be coupled to the port 640 using a threaded engagement, a friction fit, a snap fit, etc., or may be fixedly coupled using a number of techniques such as adhesive or solvent bonding. In other instances, the cap or seal may be removed through a twisting motion configured to break the cap or seal from the port 640. Once the cap or seal has been removed, the port 640 may be pierced with a spike tip or spike port adaptor (not explicitly shown) that is coupled to the gas and lens wash supply tubing 636, 638. For example, in addition to the removable cap or seal, the port 640 may include an internal seal disposed within a lumen of the port 640 that may be punctured or pierced by the spike port adaptor. The internal seal may be configured to prevent fluid 634 from leaking from the second container 632 prior to the spike port adaptor being inserted into the port 640. In some embodiments, the internal seal may be self-sealing such that upon removal of the spike port adaptor fluid is prevented from leaking from the port 640. The outer surface of the spike port adaptor may form an interference fit with the inner surface of the port 640. The fit and/or coupling between the spike port adaptor and the port 640 may be sufficient to remain in place when the gas and fluid supply tubing 636, 638 and/or other tubing sets are coupled to the spike port adaptor 610. It is contemplated that the spike port adaptor may be inserted into one of the ports 640 utilizing universally used aseptic techniques such as those used with IV fluid bags. This may help reduce infection risk by maintaining sterile components, not introducing contaminants into the fluid 606, etc. It is further contemplated that additives may be added to the fluid 606 using similar aseptic techniques via one of the ports 640, if so desired. In some cases, other coupling mechanisms may be used as desired to couple the gas and lens wash supply tubing 636, 638 to the port 640. Some illustrative coupling mechanisms may include, but are not limited to, threaded engagements, snap fits, friction fits, quick connect style couplers, etc., or may be fixedly coupled using a number of techniques such as adhesive or solvent bonding.

The gas supply tubing 636 extends from a second end external to the second container 632 to the port 640. The gas supply tubing 636 may extend into the interior of the second container 632 and terminate within a reservoir gap (e.g., above the level of the fluid 634). However, in some cases, the gas supply tubing 636 may terminate within the fluid 634. A lumen extends through the gas supply tubing 636 for receiving a flow of air and/or gas therethrough. The lumen of the gas supply tubing 636 may be in operative fluid communication with a top portion of the interior of the second container 632. The lens wash supply tubing 638 extends from a second end external to the second reservoir 630 to a first end in fluid communication with a bottom portion 642 of the second container 632. In some embodiments, the lens wash supply tubing 638 may terminate at the port 640. A lumen extends through the lens wash supply tubing 638 for receiving a flow of fluid therethrough. The lumen of the lens wash supply 638 is in selective operative fluid communication with a bottom portion 642 of the second container 632. In the illustrated embodiment, the gas supply tubing 636 and the lens wash supply tubing 638 may couple to the second container 632 through a single or common opening (e.g., port 640). For example, the gas supply tubing 636 and the lens wash supply tubing 638 may be coaxially arranged. However, this is not required. In some cases, the gas supply tubing 636 and the lens wash supply tubing 638 may extend in a side by side arrangement or may be separately connected to the second container 632 in different locations.

The second container 632 may further include a first fluid inlet 644 and a second fluid inlet 646. While the first and second fluid inlets 644, 646 are illustrated as being adjacent to or extending from a top portion 648 of the second container 632, the first and/or second fluid inlets 644, 646 may be positioned at other locations about the second container 632, as desired. In some embodiments, the first and/or second fluid inlets 644, 646 may be tubular members formed as a single monolithic structure with the second container 632. In other embodiments, the first and/or second fluid inlets 644, 646 may include tubular components releasably coupled to ports (similar in form and function to port 640) formed in or with the container 632.

The first fluid inlet 644 may be in selective fluid communication with the first reservoir 602. For example, a branched connector 650 may be positioned in-line with the upstream irrigation tubing 628. In some embodiments, the branched connector 650 may be a “Y” connector or a “T” connector having an inlet leg 656 defining a first fluid inlet, a first outlet leg 652 defining a first fluid outlet, and a second outlet leg 654 defining a second fluid outlet. However, it is contemplated that the branched connector 650 may include more than one fluid inlet and fewer than two or more than two fluid outlets, if so desired.

The branched connector 650 may be positioned in-line with the upstream irrigation tubing 628 such that the inlet leg 656 and the first outlet leg 652 are fluidly coupled with the lumen of the upstream irrigation tubing 628. Fluid may flow from the first reservoir 602, through the upstream irrigation tubing 628, through the branched connector 650 and again through the upstream irrigation tubing 628. The branched connector 650 may be positioned such that the inlet leg 656 is upstream of the outlet legs 652, 654 relative to a flow of irrigation fluid. In some embodiments, the branched connector 650 and the spike port 610 may be molded or formed as a single monolithic structure. It is contemplated that this may reduce connection points in the fluid circuit. In such an instance, the first end 620 of the irrigation supply tubing 628 may be fluidly coupled to the first outlet leg 652 of the branched connector 650.

The second outlet leg 654 may be fluidly coupled to the first fluid inlet 644 of the second reservoir 630. A flow control valve 658 may be positioned between the second fluid outlet of the second outlet leg 654 and the first fluid inlet 644 of the second reservoir 630 to selectively fluidly couple the second container 632 with the first container 604. The flow control valve 658 may be configured to be opened to allow fluid to selectively pass from the first reservoir 602 to the second reservoir 630 while preventing fluid (e.g., gas, water, or other fluid) from exiting the second container 632 and entering the irrigation supply tubing 628 and/or the first container 604. When it is desired to add fluid to the second reservoir 630 from the first reservoir 602, the flow control valve 658 may be opened or released. Fluid may then be at least partially diverted from the irrigation supply tubing 628 through the second outlet leg 654 of the branched connector 650 and into the second container 632 along flow path 660. Fluid may be added to the second container 632 while the irrigation pump 315 is running or while the irrigation pump 315 is idle, as desired.

The second fluid inlet (or gas supply tube) 646 of the second container 632 may be an alternative gas supply tubing configured to be coupled to an alternative gas supply (e.g., CO₂ hospital house gas source). The second fluid inlet 646 may extend from a second end external to the second container 632 to a first end coupled to the second container 632. The alternative gas supply may be used to pressurize the second container 632 to supply lens wash to the endoscope 100 and/or to provide insufflation. A lumen extends through the second fluid inlet 646 for receiving a flow of gas therethrough. The lumen of the second fluid inlet 646 is in operative fluid communication with a top portion of the second container 632. The flow of the CO₂ through the system 600 may be similar to that described above. For example, in the neutral state, CO₂ gas flows through the second fluid inlet 646 into the second container 632, up the gas supply tubing 636 to the connector portion 265, up the gas feed line 240b in the umbilical 260, and is vented through the gas/water valve 140 to atmosphere. In the first position, the user closes off the vent hole in the gas/water valve 140, and the CO₂ gas is flowed through the second fluid inlet 646 into the second container 632, up the gas supply tubing 636 to the connector portion 265, through the gas/water valve to the gas supply line 240a in the endoscope shaft 100a and out the gas/lens wash nozzle 220 at the distal tip 100c. In the second position, the user depresses the valve 140 to the bottom of the valve well 135, keeping the vent hole in the gas/water valve closed off. The second position blocks the CO₂ gas supply to both atmosphere and the gas supply line 240a in the endoscope 100, and opens up the gas/water valve 140 to allow lens wash water to pass through to the lens wash supply line 245a in the endoscope shaft 100a and out the gas/lens wash nozzle 220 at the distal tip 100c. Gas (pressure) in the second reservoir 630 is maintained by delivering gas through the second fluid inlet 646. It is contemplated that the flow control valve 658 is in the closed configuration during delivery of the CO₂ gas to allow the container 632 to pressurize. In some instances, the flow control valve 658 may be configured to close without user intervention in response to the delivery of CO₂ to the second container 632. In some embodiments, the system 600 may include a branched connector (such as, but not limited to a “Y” or “T” connector) at the second fluid inlet 646 to allow either air or CO₂ to be used for pressurization or insufflation. It is further contemplated that the second fluid inlet 646 may include a pressure relief valve 662, such as, but not limited to, a 3-way stopcock, a clamp, or a spring-loaded valve, to vent pressure within the second container 632 and/or to block a flow of pressurized gas to the second container 632 during refilling of the second container 632, during procedure change-overs, and/or during equipment change-overs.

It is contemplated that the use of a flexible bag in place of a rigid bottle for the second reservoir 630 may reduce or eliminate the risk of air leaking from bottle and cap connections. This may eliminate the need for clinicians to attempt to remedy the leak by adjusting the cap and bottle assemblies or from discarding a cap and/or bottle if the leak cannot be remedied.

As the pressurized second container 632 is fluidly isolated from the first container 604 when the flow control valve 658 is closed, it is contemplated that the clinician may replace the first reservoir 602 with a new (full) reservoir without losing patient insufflation. Loss of patient insufflation may result in a loss of position of the endoscope 100 within the body. In current one or two bottle systems, it may not be possible to replace the water reservoirs without loss of patient insufflation.

If there is a need to replace the first reservoir 602 with a new full bag, for example when the first reservoir 602 is empty or near empty, the user may hang the new bag near the first reservoir 602 to be replaced. The user may then disengage the spike port adaptor 610 from the port 608b and insert the spike port adaptor 610 into a port of the new bag. This may be performed without requiring the clinician to bend or stoop to access the first reservoir 602. The port 608b may self-seal to prevent fluid leaks from the first reservoir 602 being replaced. This method of replacing the first reservoir 602 may have a lower risk of introducing contaminants into the systems relative to traditional bottle systems. For example, the change out method described herein may allow the first reservoir 602 to be changed out without having tubing dangling from a cap (as in a bottle system). Further, the system 600 may remain largely closed as the first reservoir 602 is changed out.

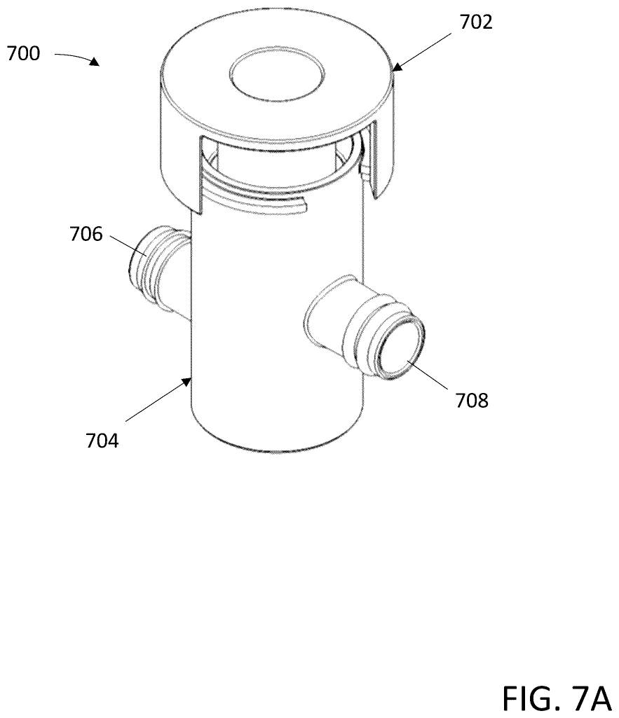

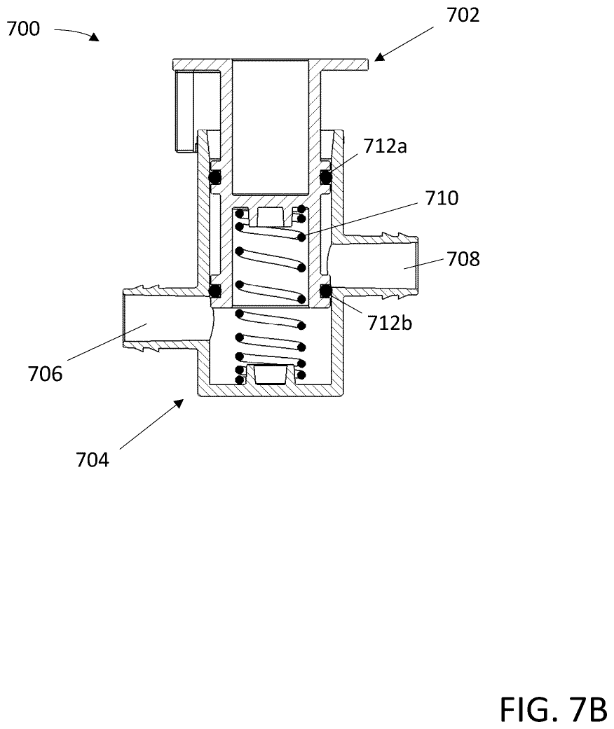

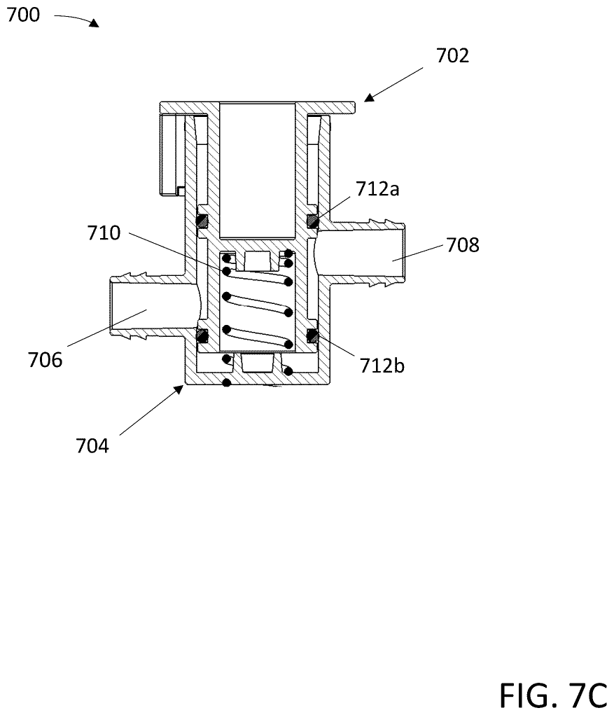

In some embodiments, the flow control valve 658 is a button valve 700 as shown in FIGS. 7A-7C. The button valve 700 includes a button 702 above a valve body 704, which is connected to the second outlet leg 654 at the valve fluid inlet 706 and fluid outlet 708.

As shown in FIG. 7B, the button valve 700 is biased by a spring member 710 into a closed position. A lower seal 712b, depicted in FIGS. 7B and 7C as an O-ring, isolates the valve fluid inlet 706 from the valve fluid outlet 708. Fluid flow is prevented in either direction—that is, fluid cannot move downstream into the pressurized chamber 648 and also cannot move upstream into the branched connector 650.

The button 702 is pressed downward by a user to move the valve into an open position as shown in FIG. 7C. In this position, an upper seal 712a is above both the fluid inlet 706 and outlet 708, while the lower seal 712b is below both the inlet and outlet. This creates a channel between the inlet 706 and outlet 708 that allows fluid to flow through the valve 700. When a user releases the button 702, the spring member 710 returns the valve 700 to the closed position.

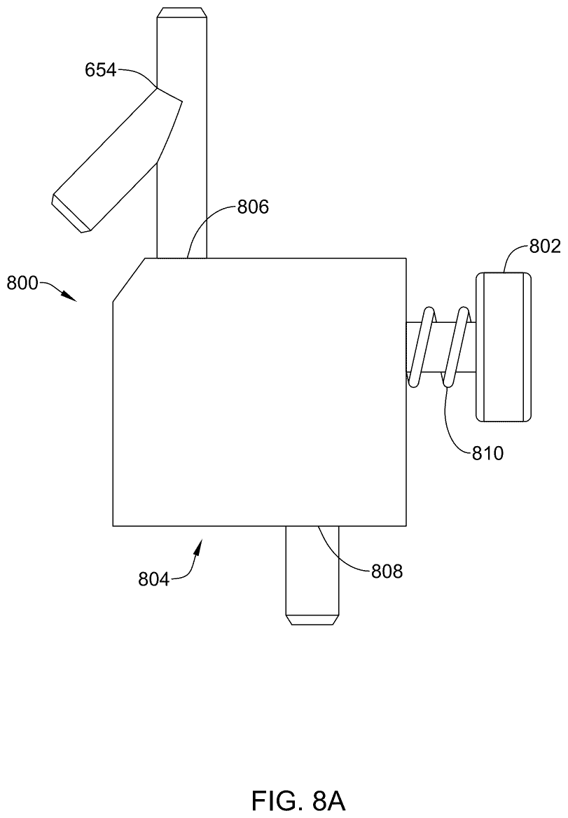

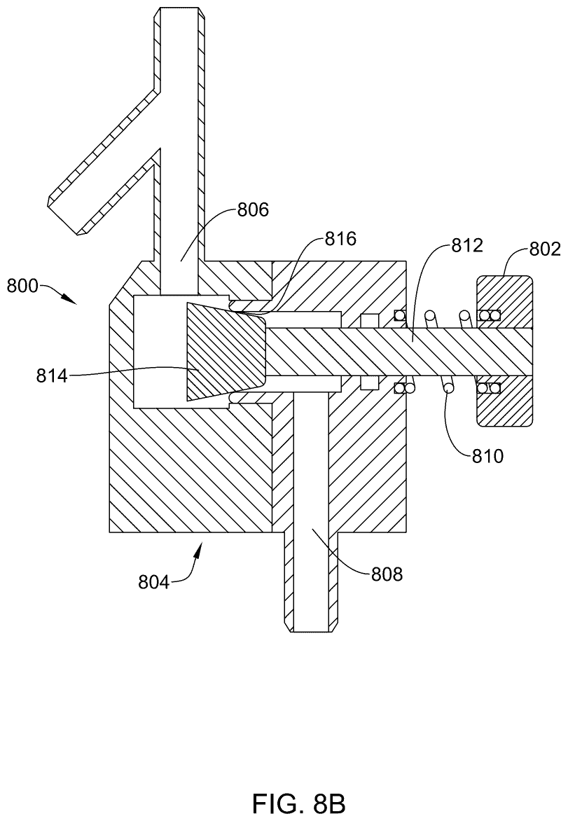

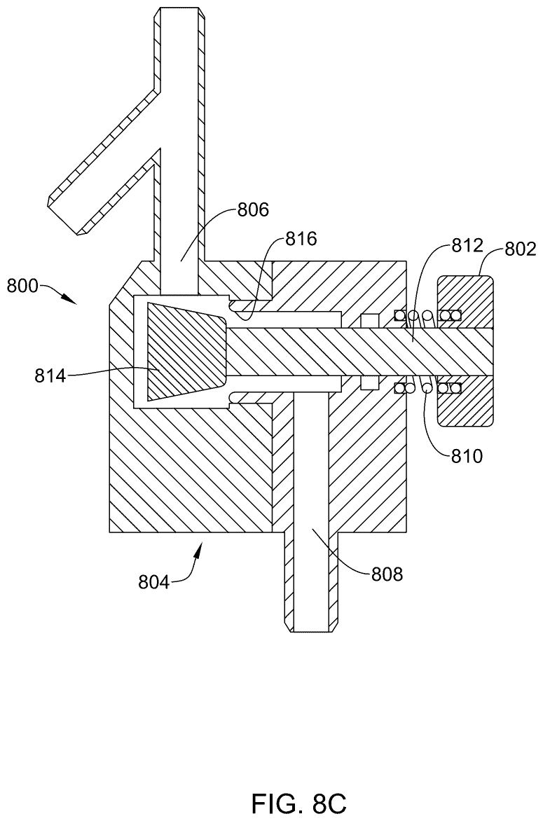

In some embodiments, the flow control valve 658 is a plug valve 800 as shown in FIGS. 8A-8C. The plug valve 800 includes a button 802 extending from a valve body 804, which is connected to the second outlet leg 654 at the valve fluid inlet 806 and fluid outlet 808. The button 802 is biased outward by a spring member 810 to open the valve 800 as shown in FIG. 8B.

Within the valve body 804, the button 802 is connected by a sliding shaft 812 to a plug member 814. The plug member 814 is seated against an inner wall 816 of the valve body 804. This prevents fluid flow between the inlet 806 and outlet 808. As shown, the plug member 814 may be conical in shape to encourage a tight seal between the plug 814 and inner wall 816, which may narrow from a first to a second diameter to better accommodate plug member. The plug member 814 may be made of a rubber or lower durometer plastic to better accommodate the seal. The biasing force of the spring member 810 holds the plug member 814 seated against the inner wall 816 to assure that the plug member 814 remains in place.

To open the valve 800, a user presses the button 802 inward, causing the plug member 814 to unseat. Fluid flows through the valve fluid inlet 806, into the valve body 804, and out through the valve fluid outlet 808. Upon releasing the button 802, the spring member 810 moves the button 802 and sliding shaft 812 outward to return the valve 800 to the closed position.

As will be appreciated, the lengths of irrigation, lens wash, gas supply, alternate gas supply tubing may have any suitable size (e.g., diameter). In addition, the sizing (e.g., diameters) of the tubing may vary depending on the application. In one non-limiting embodiment, the irrigation supply tubing may have an inner diameter of approximately 6.5 mm and an outer diameter of 9.7 mm. The lens wash supply tubing may have an inner diameter of approximately 5 mm and an outer diameter of 8 mm. The gas supply tubing may have an inner diameter of approximately 2 mm and an outer diameter of 3.5 mm. The alternative gas supply tubing may have an inner diameter of approximately 5 mm and an outer diameter of 8 mm.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

All apparatuses and methods discussed herein are examples of apparatuses and/or methods implemented in accordance with one or more principles of this disclosure. These examples are not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure.

In the foregoing description and the following claims, the following will be appreciated. The phrases “at least one”, “one or more”, and “and/or”, as used herein, are open-ended expressions that are both conjunctive and disjunctive in operation. The term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The foregoing discussion has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. One skilled in the art will appreciate that the disclosure may be used with many modifications of structure, arrangement, proportions, materials, components, and otherwise, used in the practice of the disclosure, which are particularly adapted to specific environments and operative requirements without departing from the principles of the present disclosure. For example, elements shown as integrally formed may be constructed of multiple parts or elements shown as multiple parts may be integrally formed, the operation of elements may be reversed or otherwise varied, the size or dimensions of the elements may be varied, and features and components of various embodiments may be selectively combined. The presently disclosed embodiments are therefore to be considered in all respects as illustrative and not restrictive, the scope of the claimed invention being indicated by the appended claims, and not limited to the foregoing description.

The following claims are hereby incorporated into this Detailed Description by this reference, with each claim standing on its own as a separate embodiment of the present disclosure. In the claims, the term “comprises/comprising” does not exclude the presence of other elements or steps. Furthermore, although individually listed, a plurality of means, elements or method steps may be implemented by, e.g., a single unit or processor. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second”, etc., do not preclude a plurality. Reference signs in the claims are provided merely as a clarifying example and shall not be construed as limiting the scope of the claims in any way.

What is claimed is:

1. A container and tube set arranged and configured to couple to an endoscope for use in an endoscopic procedure, the container and tube set comprising:
a first container configured to contain a fluid, the first container having a port in fluid communication with a bottom portion thereof;
a first water supply tube including a first end, a second end, and a first lumen extending therethrough, wherein the first lumen is in fluid communication with the first container and the second end of the first water supply tube is positioned external to the first container;
a branched connector positioned in line with the first water supply tube between the first and second ends thereof, the branched connector including a first fluid inlet, a first fluid outlet, and a second fluid outlet, a portion of the first lumen of the first water supply tube extending between the first fluid inlet and the first fluid outlet;
a second container configured to contain a fluid, the second container having a second fluid inlet in selective fluid communication with the second fluid outlet of the branched connector;
a flow control valve positioned to obstruct flow between the second fluid outlet of the branched connector and the second fluid inlet of the second container, the flow control valve movable between:
a closed position preventing fluid flow from the branched connector to the second container and preventing fluid flow from the second container to the branched connector, and
an open position permitting fluid flow from the branched connector to the second container; and
a second water supply tube including a first end, a second end, and a second lumen extending therethrough, wherein the second lumen is in selective fluid communication with the bottom portion of the second container and the second end of the second water supply tube is positioned external to the second container.

2. The container and tube set of claim 1, the flow control valve comprising:
a button member that moves the flow control valve into the open position while pressed, and
a spring member that biases the flow control valve to remain in the closed position until the button member is pressed and moves the flow control valve into the closed position after the button member is released from being pressed.

3. The container and tube set of claim 2, wherein the flow control valve further comprises:
a valve body, and
a plug member mechanically coupled to the button member and the spring member such that, when the button member is pressed, the plug member is unseated from the valve body, and when the button member is released, the plug member is seated against the valve body with biasing force from the spring member.

4. The container and tube set of claim 1, wherein the first container comprises a collapsible bag.

5. The container and tube set of claim 1, wherein the first container comprises a rigid bottle.

6. The container and tube set of claim 1, wherein the second container comprises a collapsible bag.

7. The container and tube set of claim 1, wherein the second container comprises a rigid bottle.

8. The container and tube set of claim 1, further comprising a first gas supply tube including a first end, a second end, and a third lumen extending therethrough, wherein the third lumen is in operative fluid communication with the second container and the second end of the first gas supply tube is positioned external to the second container.

9. The container and tube set of claim 8, further comprising a second gas supply tube including a first end, a second end, and a fourth lumen extending therethrough, wherein the fourth lumen is in operative fluid communication with the second container and the second end of the second gas supply tube is positioned external to the second container.

10. The container and tube set of claim 1, wherein the first end of the second water supply tube is fluidly coupled to a bottom portion of the second container.

11. The container and tube set of claim 1, wherein the first end of the first gas supply tube is fluidly coupled to a bottom portion of the second container.

12. An endoscopic medical device, comprising:
an endoscopic probe; and
a container and tube set arranged and configured to couple to the endoscopic probe for use in an endoscopic procedure, the container and tube set comprising:
a first container configured to contain a fluid, the first container having a port in fluid communication with a bottom portion thereof;
a first water supply tube including a first end, a second end, and a first lumen extending therethrough, wherein the first lumen is in fluid communication with the first container and the second end of the first water supply tube is positioned external to the first container;
a branched connector positioned in line with the first water supply tube between the first and second ends thereof, the branched connector including a first fluid inlet, a first fluid outlet, and a second fluid outlet, a portion of the first lumen of the first water supply tube extending between the first fluid inlet and the first fluid outlet;
a second container configured to contain a fluid, the second container having a second fluid inlet in selective fluid communication with the second fluid outlet of the branched connector;
a flow control valve positioned to obstruct flow between the second fluid outlet of the branched connector and the second fluid inlet of the second container, the flow control valve movable between:
a closed position preventing fluid flow from the branched connector to the second container and preventing fluid flow from the second container to the branched connector, and
an open position permitting fluid flow from the branched connector to the second container; and
a second water supply tube including a first end, a second end, and a second lumen extending therethrough, wherein the second lumen is in selective fluid communication with the bottom portion of the second container and the second end of the second water supply tube is positioned external to the second container.

13. The endoscopic medical device of claim 12, further comprising a pump in operative fluid communication with the first fluid outlet, the pump configured to receive fluid from the first container and deliver the received fluid to irrigation tubing in the endoscopic probe.

14. The endoscopic medical device of claim 13, wherein the pump is a peristaltic pump.

15. The endoscopic medical device of claim 12, wherein the container and tube set is further configured to deliver fluid from the second container to lens wash tubing in the endoscopic probe.

16. The endoscopic medical device of claim 12, wherein the flow control valve of the container and tube set comprises:
a button member that moves the flow control valve into the open position while pressed, and
a spring member that biases the flow control valve to remain in the closed position until the button member is pressed and moves the flow control valve into the closed position after the button member is released from being pressed.

17. The endoscopic medical device of claim 16, wherein the flow control valve of the container and tube set further comprises:
a valve body, and
a plug member mechanically coupled to the button member and the spring member such that, when the button member is pressed, the plug member is unseated from the valve body, and when the button member is released, the plug member is seated against the valve body with biasing force from the spring member.

18. The endoscopic medical device of claim 12, the container and tube set further comprising a first gas supply tube including a first end, a second end, and a third lumen extending therethrough, wherein the third lumen is in operative fluid communication with the second container and the second end of the first gas supply tube is positioned external to the second container.

19. The endoscopic medical device of claim 18, the container and tube set further comprising a second gas supply tube including a first end, a second end, and a fourth lumen extending therethrough, wherein the fourth lumen is in operative fluid communication with the second container and the second end of the second gas supply tube is positioned external to the second container.

20. The endoscopic medical device of claim 12, wherein the first container comprises a collapsible bag, and wherein the second container comprises a rigid bottle.
